(19) United States
(12) Patent Application Publication
(10) Pub. No.: US 2002/0091434 A1
(43) Pub. Date: Jul. 11, 2002

(54) Apparatus and method to position a stent

(76) Inventor: Jeffrey W. Chambers, Plymouth, MN (US)

Correspondence Address: FOSTER, SWIFT, COLLINS & SMITH, P.C., 313 SOUTH WASHINGTON SQUARE, LANSING, MI, 48933, US

(21) Appl. No.: 09/754,223

(22) Filed: Jan. 5, 2001

(51) Int. Cl. A61M 25/10

(52) U.S. Cl. 623/1.11

(57) ABSTRACT

A stent designed with an attachable positioning apparatus to effectively place the stent at the precise deployment site within a narrowed vascular region such as an artery. In the preferred embodiment, the present invention comprises a stabilizing wire. The positional apparatus is, for example, frictionally engaged to the stent balloon and adjacent wire loops for frictional engagement with the walls to position the stent at the deployment site. Other means to engage the positional apparatus and stent balloon and adjacent wire loops can include: elastic bands, adhesion, or polymer bonding. The stent is maneuvered through the vessel by a balloon catheter that is guided by a guiding catheter up the vessel to where the narrowing is located. Upon exiting the guiding catheter and approaching the deployment site within the coronary artery, the wire loops expand and frictionally engage the artery walls and, thereby, effectively position the stent at the deployment site within the narrowed vessel. This apparatus and method is particularly useful for stent placement at an ostium (origin) of a vascular region.

### BACKGROUND OF THE INVENTION

1. Field of the Invention

The present invention relates generally to an intravascular stent and, in particular, to an apparatus and method to quickly, effectively, and accurately position a stent within a stenosed (narrowed) vascular region, particularly at its ostium (origin).

2. Background And Description of The Prior Art

A stent is an intravascular prosthesis implanted in a blood vessel to maintain vascular patency in an artery, vein, lymph, or another duct in the body such as the biliary duct, ureter, or urethera (collectively referred to as vessels). For example, a stent is often a necessary treatment for atherosclerosis. Atherosclerosis is an accumulation of lipids, also known as lesions, plaques, or atheromas, in the intimal or inner layer of an affected artery. The resulting intimal thickening of lipids restricts arterial blood flow, disrupting the function of or permanently damaging the nourished organ such as the heart. Typically, the accumulation of lipids is localized and occurs in coronary, renal, cerebral, or peripheral arteries.

Treatments for atherosclerosis focus on improving blood flow through narrowed arteries. One method, balloon angioplasty, simply expands a balloon catheter to compress lipid plaque against the artery wall. Unfortunately, scar tissue (neointimal proliferation) often builds up over time and renarrows the artery. This is called restenosis. To reduce the chance of restenosis, stents are often implanted. A stent is an expandable meshed metal tube used to support a narrowed artery after angioplasty. In this procedure, the stent is deployed at the center of the lipid accumulation. Once a deployment site is identified, the stent is maneuvered through the vessel to that site. Physicians typically use fluoroscopic x-ray and injection of radiopaque contrast and marking bands on the stent balloon to determine if the stent is positioned at the narrowed region. Once positioned, the stent expands to compress the lipids, thereby opening the artery and increasing blood flow. Stenting, as described in the prior art, significantly reduces restenosis of the artery compared to balloon angioplasty alone.

Unfortunately, prior art stent positioning methods and apparatuses have several inherent shortcomings. First, the physician must rely on hand and eye coordination, using the radiopaque marking bands, to position the stent. Second, the radiopaque marking bands are generally placed either in the front of or at the back of the stent. As a result, the doctor must estimate the center position of the stent at the deployment site based on the marked stent ends. This procedure is imprecise. Third, when the stent is deployed in a coronary artery, the doctor must constantly fight the movement of a beating heart that changes the position of the artery with each inspiration and expiration. As the artery changes position, the stent position may also change. And fourth, the prior art is not able to precisely position and deploy a stent when the narrowing is at the origin (ostium) of a vessel.

Ineffective and inaccurate stent placement results in a poor overall patient outcome. If the stent is deployed too distal to the vessel narrowing, ineffective plaque compression results. Further, a higher rate of restenosis may be expected. If the stent is placed too proximal to a narrowing at the aorta origin (ostium), the stent may hang into the aorta and a thrombus (clot) may form on the stent. Placement of the stent too proximal may also result in inappropriate and unintended blockage of another blood vessel.

Thus, an apparatus and method is needed to more effectively and accurately position a stent at a desired deployment site within the narrowed area of a vessel, thereby improving overall patient outcome.

### SUMMARY OF THE INVENTION

Accordingly, the present invention provides an apparatus and method to effectively position a stent within an intravascular region (vessel) that has narrowed, including the ostium (origin) of the vessel. Additional and related objects of the present invention include: providing a more efficient device to position the stent at a deployment site; reducing the number of stents required to compress the plaque and open the vessel to abate the reduction in blood flow caused by the occlusion; preventing restenosis; reducing the amount of time needed to position the stent within the narrowed vessel, thereby reducing the amount of time required to perform the procedure; reducing the amount of time that a patient must remain in the cardiac catheterization lab; reducing radiation exposure to the patient and cardiac catheterization staff; reducing the amount of radiopaque contrast used, thereby decreasing the risk of renal failure; minimizing the costs to repair and strengthen a narrowed vessel; and reducing heart movement as a stenting obstacle, whereby the stent maintains position within a narrowed coronary artery prior to deployment.

The present invention comprises a positional apparatus for a stent. The positional apparatus can be frictionally engaged to the distal end of the stent, stent balloon or stent catheter. Other possible ways to attach the apparatus to the stent may include elastic bands, springs, adhesives, welds, clasps, screws, snaps, magnets, polymer bondings, or by contiguous formation with the stent, stabilizing wire or a balloon catheter. The apparatus has an expandable and retractable element used to engage the vessel wall to position the stent at the deployment site. The apparatus and stent are maneuvered through the vessel by a balloon catheter that is guided by a guiding wire and guiding catheter up the vessel to the narrowing. Upon exiting the guiding catheter and approaching the deployment site within the narrowed vessel, the positional element is expanded, thereby engaging the wall of the vessel and positioning the stent at the precise deployment site within the narrowed vessel.

Other objects of the present invention will become more apparent to persons having ordinary skill in the art to which the present invention pertains from the following description taken in conjunction with the accompanying drawings.

### BRIEF DESCRIPTION OF THE FIGURES

The foregoing objects, advantages, and features of the present invention, as well as other objects and advantages, will become apparent with reference to the description and drawings below, in which like numerals represent like elements and in which:

FIG. 1 is a side elevational view of a positional apparatus for a stent.

FIG. 2 is a front cross-sectional view of the positional apparatus illustrating one of the preferred embodiments of the present invention with a connector (spring) expandable element (wire loops).

FIG. 3 is a side elevational view of a stabilizing wire attached to the positional apparatus and stent and coaxially mounted within a balloon catheter.

FIG. 4 is a front view of a patient illustrating insertion of the stent into the human body and the direction the stent follows to a narrowed vascular region.

FIG. 5 is a side cross-sectional view of a vessel with the positional apparatus and stent at the vessel deployment site.

FIG. 6 is a front cross-sectional view, taken along line 6-6 of FIG. 5, of the narrowed vessel in relation to the positional device, stent and the Stabilizing Wire.

FIG. 7 is a side cross-sectional view of the expanded balloon catheter and stent being deployed within a vessel.

FIG. 8 is a side cross-sectional view of the deployed stent and the removal of the stent placement device, Stabilizing Wire, and catheter.

FIG. 9 is a side perspective view of the stent as deployed within the narrowed vessel.

FIG. 10 shows a side perspective view of the stent, catheter balloon, and positional device while moving toward the narrowed vascular region.

FIG. 11 shows a side perspective view of the catheter balloon and positional device while moving away from the stented vascular region.

FIG. 12 shows an alternative preferred embodiment of the present invention using a new type of balloon catheter with an annular ring.

FIG. 13 shows an alternative preferred embodiment of the present invention using a flange as the deployment site regulator.

FIG. 14 shows an alternative preferred embodiment of the present invention using rods as the deployment site regulator.

### DETAILED DESCRIPTION OF THE PREFERRED EMBODIMENT

The present invention generally relates to a device and method for stent placement. Although the preferred embodiment describes use within an artery, the invention could be applied to any region of a person or animal where stent placement is used to open a narrowed vessel. Please note that proximal and distal orientation relationships in this description are in relationship to their point of insertion in a body and not the orientation about the narrowing of the vessel. This includes orientations of the stent apparatus itself where the proximal stent end is actually what many physicians refer to as the distal stent end.

FIG. 1 illustrates a stent positioning apparatus 20 in one of its preferred embodiments and is not intended to limit the apparatus in any way. The preferred stent positioning apparatus 20 consists of a plurality of stabilizing wires 22 and a deployment site regulator 24. The stabilizing wires 22 have a proximal end 26 and a distal end 28. Proximal end 26 and distal end 28 are separated by a stabilizing wire length 30 with a stabilizing wire spacing diameter 32. The stabilizing wire length 30 is of sufficient length to reach a narrowed region within either a primary blood vessel or a coronary artery. Preferably, the stabilizing wire length 30 is approximately 160 centimeters long and the stabilizing wire spacing diameter 32 is approximately 0.3 centimeters wide.

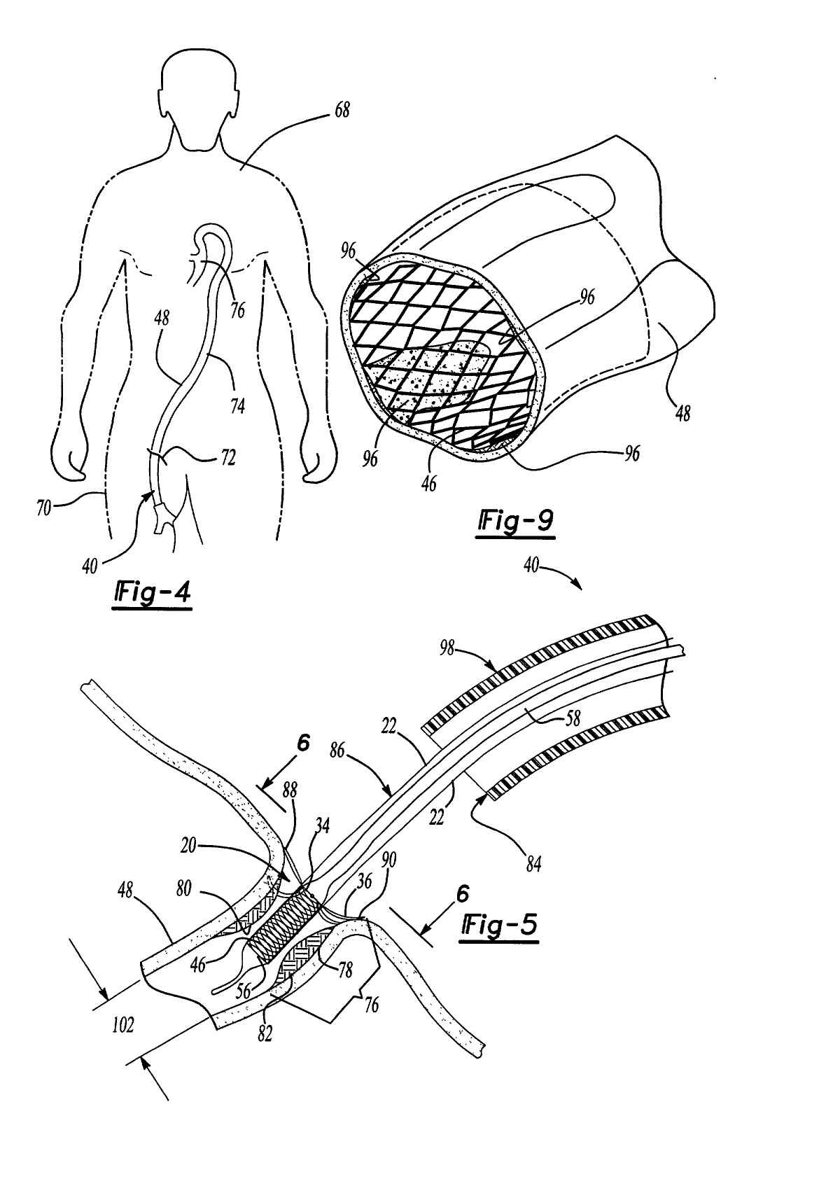

The deployment site regulator 24 is attached to the distal end 28 of the stabilizing wires 22. Attachment can be by means of frictional engagement, elastic bands, springs, adhesives, welds, clasps, screws, snaps, magnets, polymer bondings, or contiguous with any stent placement element such as a stent 46, a balloon catheter 54 or the stabilizing wires 22. In the preferred embodiment, the deployment site regulator 24 comprises a spring 34 and a plurality of wire loops 36. The spring 34 is attached to the stabilizing wires 22 such that the spring 34 and stabilizing wires 22 will not become detached during a stent procedure. The wire loops 36 extend outwardly from and parallel to spring 34. The wire loops 36 are attached to spring 34 at an attachment point 38. Preferably, wire loops 36 are permitted unrestricted rotation about attachment point 38 in relation to spring 34. The unrestricted rotation allows wire loops 36 to be maneuvered through a guiding catheter 40, having a guiding catheter sheath 98 (FIG. 5), to or from the narrowed region of the vessel during the stent procedure. Wire loops 36 have a surface such that contact with the interior walls of guiding catheter 40 does not impede the progress of wire loops 36 through guiding catheter 40, but is sufficient to frictionally engage a vessel 48 interior wall (FIG. 4) without damaging the vessel 48.

In FIG. 5, vessel 48 has a vessel diameter 102 that is sufficiently smaller than a wire loop diameter 104 (FIG. 6) in its natural position. Preferably, wire loops 36 are made of a nitinol wire frame. Alternatively, the wire loops 36 may be made of another type of wire frame or material, provided the wire loops 36 made with the alternative material are able to perform the same functions as the wire loops 36 with the nitinol wire. A further embodiment, described below and shown in FIG. 12, removes the wire loops 36 and instead uses a balloon catheter with an expanded diameter annular ring to engage adjacent structures of the vessel 48.

Other embodiments of the deployment site regulator 24 could include any device that can make its way through the guiding catheter sheath 98 in a retracted position, but can expand to engage adjacent structures of the vessel 48. These could include a rubber flange 108 (FIG. 13) or outward radiating rods 110 (FIG. 14) instead of loops.

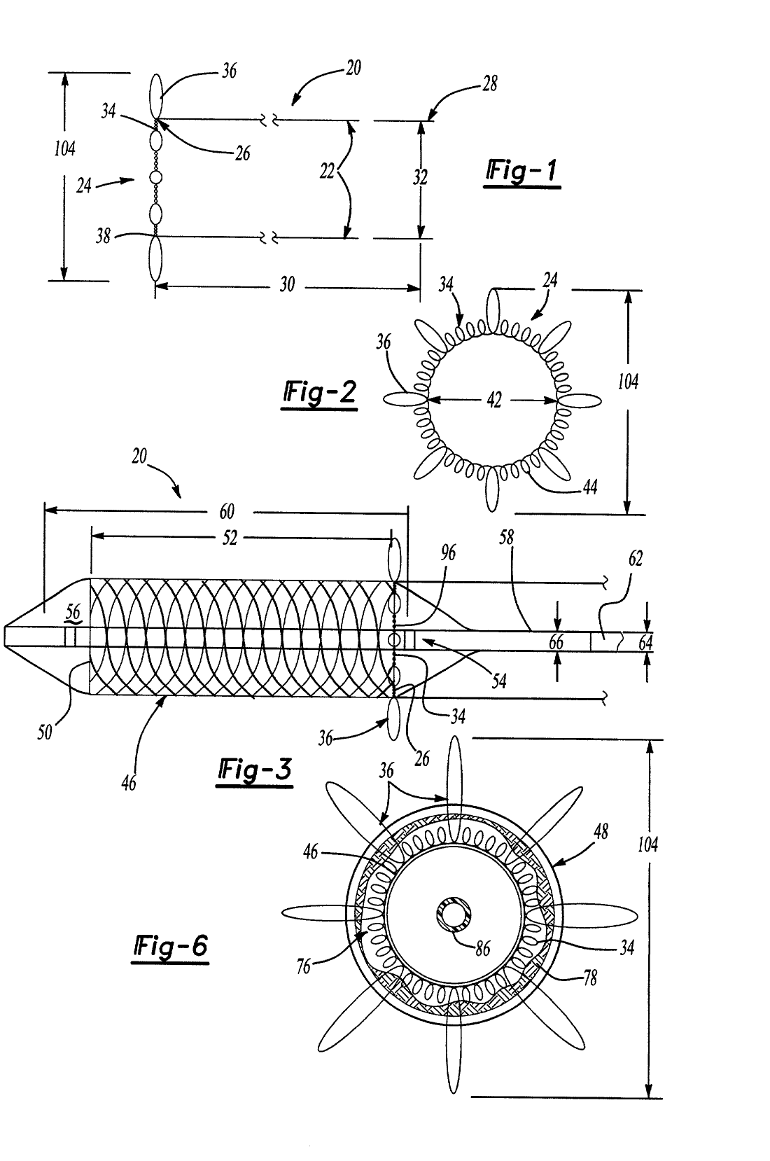

FIG. 2 illustrates the deployment site regulator 24 with more specificity. Preferably, the stabilizing wires 22 and spring 34 are circular in shape and the spring 34 has a diameter 42 approximately equal to the stabilizing wire spacing diameter 32. Spring 34 is a closed loop that consists of a plurality of coils 44. The coils 44 are situated adjacent to one another in equal spacing around the entire periphery of spring 34. Coils 44 permit spring 34 to be expanded during the stent procedure. Wire loops 36 are attached to spring 34 between coils 44. Preferably, wire loops 36 are equally spaced around the entire periphery of spring 34. FIG. 2 is a non-limiting example which depicts eight wire loops 36 around the entire periphery of spring 34. Alternatively, the deployment site regulator 24 may contain more or fewer wire loops 36 as long as the proper frictional engagement is provided by wire loops 36 to accurately position the stent 46 (FIG. 3) at the deployment site within the vessel 48. The deployment site regulator 24 is releasably attached to the stent 46 using a frictional engagement. As the stent 46 expands to the deployed position, the deployment site regulator 24 is released from the stent 46. Elastic bands, springs, adhesives, welds, clasps, screws, snaps, magnets, polymer bondings, or contiguous with the stent 46 or the stabilizing wires 22 can be used rather than frictional engagement to releasably attach the stent 46 to the deployment site regulator 24. In an alternative embodiments shown below, a specially shaped catheter is described.

The interconnection of stent 46 to stabilizing wires 22 is more clearly illustrated in FIG. 3. Stent 46 is generally a hollow, cylindrical prosthesis that comprises thin walled, tubular members that define a narrow web-like mesh. Stent 46 has a stent proximal end 50 and a stent distal end 96. Stent distal end 96 of stent 46 is attached to spring 34 at proximal end 26 of the stabilizing wires 22. Stent 46 has a stent length 52 of approximately eight to thirty-eight millimeters. Extending throughout the hollow center of stent 46 and stabilizing wires 22 is the balloon catheter 54. Balloon catheter 54 comprises a balloon 56 and a balloon shaft 58. Balloon 56 is releasably mounted and centered within stent 46 and has a balloon length 60. The balloon length 60 will correspond to the stent length 52 and has an overhand of approximately 0.1-0.2 millimeters beyond the stent 46. Alternatively, the balloon length 60 may be equal to or smaller than the stent length 52 as long as balloon 56 is capable of inflating to effectively expand stent 46. Within balloon 56 is a balloon guidewire 62. Balloon guidewire 62 has a balloon guidewire diameter 64. In the preferred embodiment, balloon guidewire diameter 64 is 0.14 centimeters.

Balloon shaft 58 has a balloon shaft diameter 66. In the preferred embodiment, balloon shaft diameter 66 is approximately 0.8 millimeters.

In FIG. 4, a non-limiting example of the inventive apparatus is depicted in which guiding catheter 40 is inserted into a human body 68. Typically, guiding catheter 40 is inserted or cannulated into the vessel 48 which is located in a leg 70 of the human body 68. A portion of guiding catheter 40 remains outside of the human body 68 while the remainder of guiding catheter 40 is inserted into human body 68. Guiding catheter 40 enters human body 68 at an incision point 72 and follows through vessel 48 along a path 74. Vessel 48, at incision point 72, is a femoral artery that becomes an iliac artery and then the aorta artery at the point where the iliac arteries merge. Guiding catheter 40 follows path 74 until it reaches a point near the primary coronary arteries of a heart where a narrowed vascular region 76 is located.

In FIG. 5, the proximal portion of vessel 48 is enlarged to depict its origin and the positioning of stent 46 within the narrowed vascular region 76, the site of deployment. Narrowed vascular region 76 consists of an accumulation of lipids 78 that form large patches (atherosclerotic plaques) 80 and 82 on the interior walls of vessel 48. In many instances, patch 80 almost contacts patch 82. Narrowed vascular region 76 represents the location of the highest concentration of lipids 78 in which patches 80 and 82 restrict the greatest amount of blood flow through vessel 48.

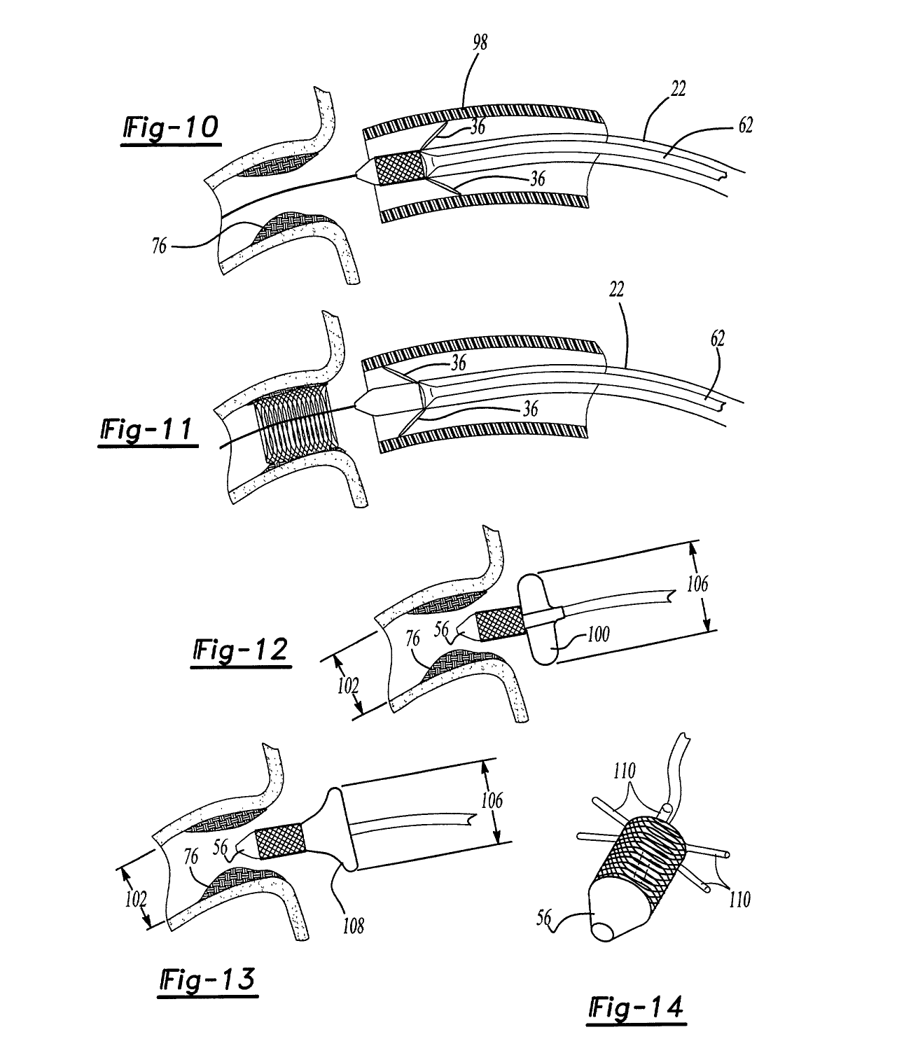

To perform the stent procedure, guiding catheter 40, as explained earlier, is first inserted into human body 68 and manipulated through vessel 48 to a holding position 84 near the entry of vessel 48 and the narrowed vascular region 76. Next, stent 46 and balloon 56 are connected to the stent positioning apparatus 20 outside the human body 68. A stent-balloon catheter combination 86 with the stent positioning apparatus 20 attached is inserted into and manipulated through the guiding catheter sheath 98. During the manipulation through guiding catheter sheath 98, wire loops 36 contact the interior wall and are forced into a rearward trailing position with respect to stent 46, as illustrated in FIG. 10. Stent-balloon catheter combination 86 exits guiding catheter 40 at holding position 84. Upon exiting guiding catheter 40, wire loops 36 return to an approximately perpendicular position with respect to stent 46. The stent-balloon catheter combination 86 is then manipulated toward narrowed vascular region 76. Upon nearing narrowed vascular region 76, the target deployment site, the wire loops 36 begin to frictionally engage the adjacent walls of vessel 48 at engagement points 88 and 90. The frictional engagement of the wire loops 36 with the walls of vessel 48 adjacent to the deployment site suspends the forward movement of stent-balloon catheter combination 86 through vessel 48. The forward movement of stent-balloon catheter combination 86 is suspended at the deployment site and stent-balloon catheter combination 86 is centered directly within narrowed vascular region 76 as illustrated more clearly in the cross-sectional view of FIG. 6.

FIG. 6 illustrates the accumulation of lipids 78 around the entire interior periphery of vessel 48 with stent-balloon catheter combination 86 located in the center of vessel 48 at the deployment site. Once stent-balloon catheter combination 86 is positioned within the deployment site of narrowed vascular region 76, balloon 56 is inflated, as illustrated in FIG. 7. When balloon 56 begins to inflate, the exterior of balloon 56 contacts the interior of stent 46 and outwardly forces stent 46 into an expanded position. As stent 46 expands with the inflation of balloon 56, spring 34 correspondingly expands with stent 46 to expansion points 92 and 94 in FIG. 7. As balloon 56 inflates, it applies pressure on lipids 78. Since lipids 78 are a waxy type material, lipids 78 succumb to the pressure of balloon 56 and, thereby, compress against the walls of vessel 48. The compression of lipids 78 reduces the blockage and expands the diameter of vessel 48 to restore vessel patency or blood flow through vessel 48.

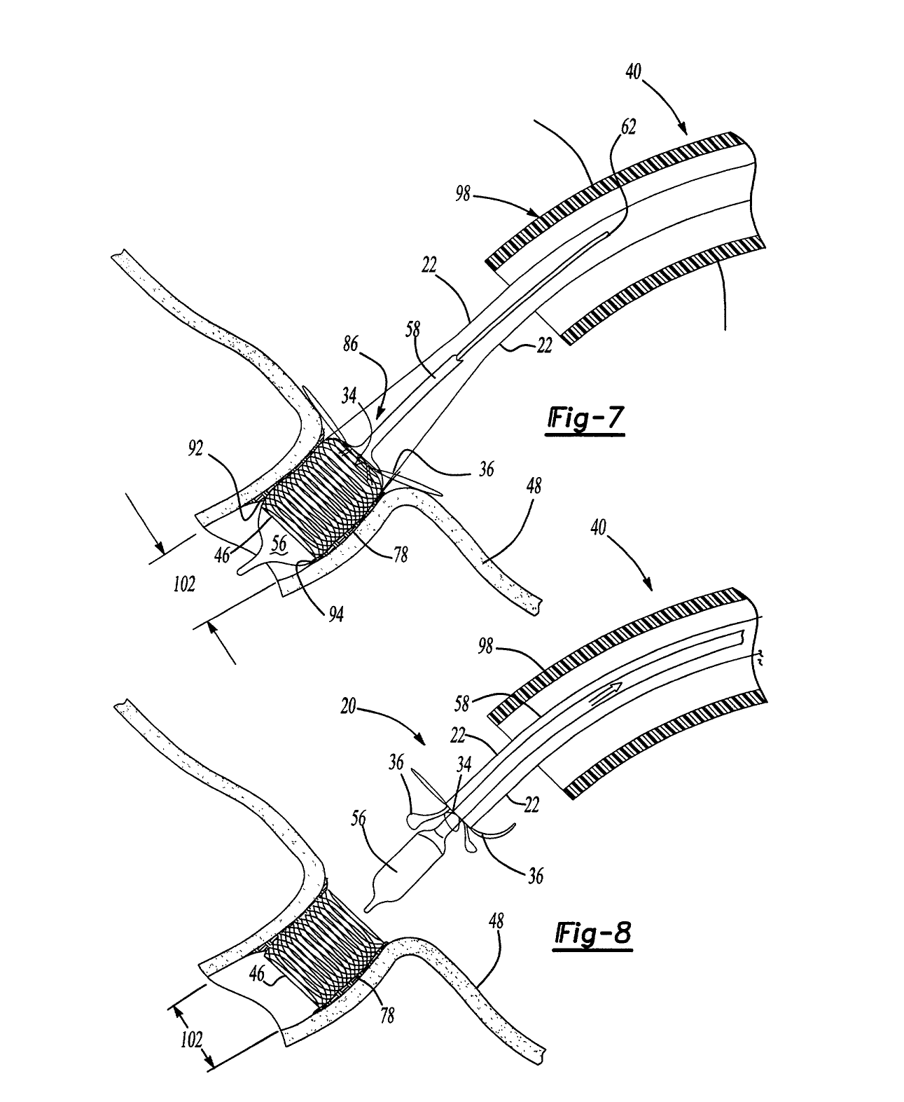

In FIG. 8, with the stent 46 in place, balloon 56 is deflated and balloon catheter 54 with the stent positioning apparatus 20 is retracted through the guiding catheter 40 for removal from human body 68. Guiding catheter 40 is then removed from human body 68. FIG. 11 illustrates the removal of the balloon catheter 54 and the stent positioning apparatus 20. As before, wire loops 36 contact the interior wall of vessel 48 and are forced into a rearward trailing position with respect to the direction of removal. Stent 46 remains within vessel 48, as illustrated in FIG. 9, as a prosthesis to repair or strengthen vessel 48 and prevent restenosis.

As an alternative preferred embodiment of the present invention, FIG. 12 illustrates a specially designed balloon catheter instead of the plurality of wire loops 36 to hold the stent 46 in place. As shown, prior to reaching the narrowed vascular region 76, an annular ring balloon 100 is inflated until an annular ring balloon diameter 106 sufficiently exceeds the vessel diameter 102 to suspend the forward movement of the stent-balloon catheter combination 86 through vessel 48. This embodiment could also be achieved using only one balloon catheter. This specially shaped catheter would be partially inflated before reaching the narrowed vascular region 76 so that the stent-balloon catheter combination 86 portion of the catheter was smaller in diameter than vessel diameter 102, including narrowed vascular region 76. But, the annular ring balloon 100 portion would, as before, have an annular ring balloon diameter 106 sufficiently exceeding the vessel diameter 102 to suspend the forward movement of the stent-balloon catheter combination 86 through vessel 48.

Thus, an apparatus and method is provided to guide the placement of a stent within the deployment site of a narrowed blood vessel for accurate deployment during a stent procedure. While the invention has been described in conjunction with specific embodiments, it is evident that many alternatives, modifications and variations will be apparent to those skilled in the art in light of the foregoing description. Accordingly, the present invention attempts to embrace all such alternatives, modifications and variations that fall within the spirit and scope of the appended claims.

1. A stent placement device comprising:
a guiding catheter having a hollow bore and proximal and distal ends and at least one stabilizing wire threaded through the hollow bore having proximal and distal ends;
a stent, with proximal and distal ends, inflation expandable from a delivery diameter to a deployment diameter, such that the delivery diameter is reduced from the deployment diameter to allow passage through the guiding catheter, such that the stent, in its delivery diameter, is coaxially mounted within the guiding catheter near the catheter distal end and attached to a balloon catheter;
the balloon catheter coaxially positioned within the guiding catheter for expansion of the stent at a deployment site from the delivery diameter to the deployment diameter upon application of deployment pressure to a balloon catheter expandable inflation means;
the balloon catheter comprising a shaft and the expandable inflation means being positioned at a distal part of the shaft, a releasably mounting and retaining means being positioned for receiving the stent on the expandable inflation means for radial expansion and release of the stent upon expansion of the expandable inflation means;
a deployment site regulator, comprising a means to attach the balloon catheter and the distal end of the stabilizing wire and an expandable positioning means to allow the stent to be positioned at the deployment site by frictionally engaging adjacent structures near the deployment site, the expandable positioning means having a pre-deployment diameter to allow passage through the guiding catheter, and the deployment diameter to provide support to engage the adjacent deployment site structures after passage through the guiding catheter.

2. The stent placement device of claim 1, wherein the deployment site regulator further comprises a means to attach to the proximal stent end.

3. The stent placement device of claim 1, wherein the deployment site regulator further comprises a means to attach to the guiding catheter.

4. The stent placement device of claim 1, wherein the deployment site regulator attachment means is by frictional engagement.

5. The stent placement device of claim 1, wherein the deployment site regulator attachment means is by elastic bands.

6. The stent placement device of claim 1, wherein the deployment site regulator attachment means is by springs.

7. The stent placement device of claim 1, wherein the deployment site regulator attachment means is by adhesives.

8. The stent placement device of claim 1, wherein the deployment site regulator attachment means is by welds.

9. The stent placement device of claim 1, wherein the deployment site regulator attachment means is by clasps.

10. The stent placement device of claim 1, wherein the deployment site regulator attachment means is by screws.

11. The stent placement device of claim 1, wherein the deployment site regulator attachment means is by snaps.

12. The stent placement device of claim 1, wherein the deployment site regulator attachment means is by magnets.

13. The stent placement device of claim 1, wherein the deployment site regulator attachment means is by polymer bonding.

14. The stent placement device of claim 1, wherein the deployment site regulator attachment means is by contiguous formation with the stent.

15. The stent placement device of claim 1, wherein the deployment site regulator attachment means is by contiguous formation with the balloon catheter.

16. The stent placement device of claim 1, wherein the deployment site regulator attachment means is by contiguous formation with the stabilizing wire.

17. The stent placement device of claim 1, wherein the deployment site regulator is the balloon catheter further comprising an annular ring as part of the expandable inflation means;
the annular ring positioned for releasably mounting and extending distally from the distal stent end for radial expansion, whereby an expanded annular ring diameter provides support to engage adjacent deployment site structures and release the stent upon expansion of the expandable inflation means.

18. The stent placement device of claim 1, wherein the deployment site regulator is a second balloon catheter, the second balloon catheter having a second expandable inflation means comprising an annular ring;
the second balloon catheter coaxially positioned within the guiding catheter having a second balloon catheter delivery diameter for passage through the guiding catheter to a second balloon catheter deployment diameter upon application of deployment pressure to the second expandable inflation means;
and the second balloon catheter comprising a second shaft and the expandable inflation means being positioned at a distal part of the second shaft and proximal to the balloon catheter, a mounting and retaining means being positioned for releasably mounting and extending proximally from the proximal stent end for radial expansion of the annular ring when the balloon catheter is inflated and the stent released, whereby an expanded annular ring diameter provides support to engage adjacent deployment site structures and release the stent upon expansion of the expandable inflation means.

19. The stent placement device of claim 1, wherein the deployment site regulator comprises a flange shaped device.

20. The stent placement device of claim 1, wherein the deployment site regulator comprises at least two outward radiating rods.

21. The stent placement device of claim 1, wherein the deployment site regulator comprises a plurality of flexible loops, whereby the loops can compress to the pre-deployment diameter during passage through the guiding catheter and return naturally to its expanded state when not compressed or restricted.

22. The stent placement device of claim 21, wherein the the plurality of flexible loops are wire.

23. The stent placement device of claim 21, wherein the the plurality of flexible loops are nitinol wire.

24. The stent placement device of claim 21, wherein the the plurality of flexible loops are plastic.

25. The stent placement device of claim 21, wherein the the plurality of flexible loops are a polymer.

26. The stent placement device of claim 21, wherein the the plurality of flexible loops are nylon.
